(19) United States
(12) Patent Application Publication
(10) Pub. No.: US 2026/0262750 A1
(43) Pub. Date: Sep. 10, 2026

(54) SMOKING SUBSTITUTE SYSTEM

(71) Applicant: Imperial Tobacco Limited, Bristol (GB)

(72) Inventors: Chris Lord, Bristol (GB); Med Benyezzar, Bristol (GB); Roland Zitzke, Bristol (GB)

(21) Appl. No.: 19/396,242

(22) Filed: Nov. 20, 2025

Related U.S. Application Data
(63) Continuation of application No. 17/481,864, filed on Sep. 22, 2021, now Pat. No. 12,501,942.
Application No. 17/481,864 is a continuation of application No. PCT/EP2020/056783, filed on Mar. 13, 2020.
Application No. PCT/EP2020/056783 is a continuation of application No. PCT/EP2020/056790, filed on Mar. 13, 2020.
Application No. PCT/EP2020/056790 is a continuation of application No. PCT/EP2020/056794, filed on Mar. 13, 2020.
Application No. PCT/EP2020/056794 is a continuation of application No. PCT/EP2020/056795, filed on Mar. 13, 2020.
Application No. PCT/EP2020/056795 is a continuation of application No. PCT/EP2020/056798, filed on Mar. 13, 2020.
Application No. PCT/EP2020/056798 is a continuation of application No. PCT/EP2020/056800, filed on Mar. 13, 2020.
Application No. PCT/EP2020/056800 is a continuation of application No. PCT/EP2020/056801, filed on Mar. 13, 2020.
Application No. PCT/EP2020/056801 is a continuation of application No. PCT/EP2020/056803, filed on Mar. 13, 2020.
Application No. PCT/EP2020/056803 is a continuation of application No. PCT/EP2020/056804, filed on Mar. 13, 2020.
Application No. PCT/EP2020/056804 is a continuation of application No. PCT/EP2020/056810, filed on Mar. 13, 2020.
Application No. PCT/EP2020/056810 is a continuation of application No. PCT/EP2020/056812, filed on Mar. 13, 2020.
Application No. PCT/EP2020/056812 is a continuation of application No. PCT/EP2020/056817, filed on Mar. 13, 2020.
Application No. PCT/EP2020/056817 is a continuation of application No. PCT/EP2020/056827, filed on Mar. 13, 2020.
Application No. PCT/EP2020/056827 is a continuation of application No. PCT/EP2020/056833, filed on Mar. 13, 2020.
Application No. PCT/EP2020/056833 is a continuation of application No. PCT/EP2020/056834, filed on Mar. 13, 2020.
Application No. PCT/EP2020/056834 is a continuation of application No. PCT/EP2020/056835, filed on Mar. 13, 2020.
Application No. PCT/EP2020/056835 is a continuation of application No. PCT/EP2020/056841, filed on Mar. 13, 2020.
Application No. PCT/EP2020/056841 is a continuation of application No. PCT/EP2020/056852, filed on Mar. 13, 2020.

(30) Foreign Application Priority Data
Mar. 22, 2019 (EP) 19020139.2
Mar. 22, 2019 (EP) 19020144.2
Mar. 22, 2019 (EP) 19020145.9
Mar. 22, 2019 (EP) 19020146.7
Mar. 22, 2019 (EP) 19020162.4
Mar. 22, 2019 (EP) 19020174.9
Mar. 22, 2019 (EP) 19020192.1
Mar. 22, 2019 (EP) 19020196.2
Mar. 22, 2019 (EP) 19020198.8
Mar. 22, 2019 (EP) 19020202.8
Mar. 22, 2019 (EP) 19020215.0
Mar. 22, 2019 (EP) 19020217.6
Mar. 22, 2019 (EP) 19020221.8
Mar. 22, 2019 (EP) 19020222.6
Mar. 22, 2019 (EP) 19020224.2
Mar. 22, 2019 (EP) 19020226.7
Mar. 22, 2019 (EP) 19020227.5
Mar. 22, 2019 (EP) 19020228.3

(51) Int. Cl. A24F 40/90; A24F 40/20; A24F 40/51; A24F 40/53; A24F 40/57; A24F 40/60; A24F 40/65; A61M 15/06; H02J 7/80

(52) U.S. Cl. CPC A24F 40/90; A24F 40/20; A24F 40/51; A24F 40/53; A24F 40/57; A24F 40/60; A24F 40/65; H02J 7/80; A61M 15/06

(57) ABSTRACT

A smoking substitute device and a smoking substitute system comprising the device are described. The device includes a controller configured to detect an error event and a user misuse event.

### CROSS REFERENCE TO RELATED APPLICATIONS/INCORPORATION BY REFERENCE STATEMENT

This application is a continuation of United States nonprovisional utility patent application Ser. No. 17/481,864, filed on 22 Sep. 2021 and issued on 23 Dec. 2025 as U.S. Pat. No. 12,501,942B2 , which is a continuation of the following international patent applications: no. PCT/EP2020/056783, filed on 13 Mar. 2020, which claims priority to EP 19020145.9, filed on 22 Mar. 2019; no. PCT/EP2020/056794, filed on 13 Mar. 2020, which claims priority to EP 19020215.0, filed on 22 Mar. 2019; no. PCT/EP2020/056812, filed on 13 Mar. 2020, which claims priority to EP 19020146.7, filed on 22 Mar. 2019; no. PCT/EP2020/056795, filed on 13 Mar. 2020, which claims priority to EP 19020222.6, filed on 22 Mar. 2019; no. PCT/EP2020/056798, filed on 13 Mar. 2020, which claims priority to EP 19020226.7, filed on 22 Mar. 2019; no. PCT/EP2020/056800, filed on 13 Mar. 2020, which claims priority to EP 19020228.3, filed on 22 Mar. 2019; no. PCT/EP2020/056790, filed on 13 Mar. 2020, which claims priority to EP 19020162.4, filed on 22 Mar. 2019; no. PCT/EP2020/056817, filed on 13 Mar. 2020, which claims priority to EP 19020144.2, filed on 22 Mar. 2019; no. PCT/EP2020/056827, filed on 13 Mar. 2020, which claims priority to EP 19020192.1, filed on 22 Mar. 2019; no. PCT/EP2020/056803, filed on 13 Mar. 2020, which claims priority to EP 19020139.2, filed on 22 Mar. 2019; no. PCT/EP2020/056833, filed on 13 Mar. 2020, which claims priority to EP 19020198.8, filed on 22 Mar. 2019; no. PCT/EP2020/056801, filed on 13 Mar. 2020, which claims priority to EP 19020196.2, filed on 22 Mar. 2019; no. PCT/EP2020/056834, filed on 13 Mar. 2020, which claims priority to EP 19020217.6, filed on 22 Mar. 2019; no. PCT/EP2020/056804, filed on 13 Mar. 2020, which claims priority to EP 19020174.9, filed on 22 Mar. 2019; no. PCT/EP2020/056810, filed on 13 Mar. 2020, which claims priority to EP 19020202.8, filed on 22 Mar. 2019; no. PCT/EP2020/056835, filed on 13 Mar. 2020, which claims priority to EP 19020224.2, filed on 22 Mar. 2019; no. PCT/EP2020/056841, filed on 13 Mar. 2020, which claims priority to EP 19020227.5, filed on 22 Mar. 2019; and no. PCT/EP2020/056852, filed on 13 Mar. 2020, which claims priority to EP 19020221.8, filed on 22 Mar. 2019. The entire contents of each of the above-referenced applications and of the above-referenced issued Unites States patent are hereby incorporated herein by reference in their entirety.

### TECHNICAL FIELD

The present invention relates to a smoking substitute system and particularly, although not exclusively, to a smoking substitute system comprising a device configured to detect an error event of the device and an event of user misuse of the device, and methods of detecting an error event and an event of user misuse of the device.

### BACKGROUND

The smoking of tobacco is generally considered to expose a smoker to potentially harmful substances. It is generally thought that a significant amount of the potentially harmful substances are generated through the heat caused by the burning and/or combustion of the tobacco and the constituents of the burnt tobacco in the tobacco smoke itself.

Conventional combustible smoking articles, such as cigarettes, typically comprise a cylindrical rod of tobacco comprising shreds of tobacco which is surrounded by a wrapper, and usually also a cylindrical filter axially aligned in an abutting relationship with the wrapped tobacco rod. The filter typically comprises a filtration material which is circumscribed by a plug wrap. The wrapped tobacco rod and the filter are joined together by a wrapped band of tipping paper that circumscribes the entire length of the filter and an adjacent portion of the wrapped tobacco rod. A conventional cigarette of this type is used by lighting the end opposite to the filter and burning the tobacco rod. The smoker receives mainstream smoke into their mouth by drawing on the mouth end or filter end of the cigarette.

Combustion of organic material such as tobacco is known to produce tar and other potentially harmful by-products. There have been proposed various smoking substitute systems (or “substitute smoking systems”) in order to avoid the smoking of tobacco.

Such smoking substitute systems can form part of nicotine replacement therapies aimed at people who wish to stop smoking and overcome a dependence on nicotine.

Smoking substitute systems include electronic systems that permit a user to simulate the act of smoking by producing an aerosol (also referred to as a “vapor”) that is drawn into the lungs through the mouth (inhaled) and then exhaled. The inhaled aerosol typically bears nicotine and/or flavorings without, or with fewer of, the odor and health risks associated with traditional smoking.

In general, smoking substitute systems are intended to provide a substitute for the rituals of smoking, whilst providing the user with a similar experience and satisfaction to those experienced with traditional smoking and with combustible tobacco products. Some smoking substitute systems use smoking substitute articles (also referred to as a “consumables”) that are designed to resemble a traditional cigarette and are cylindrical in form with a mouthpiece at one end.

The popularity and use of smoking substitute systems has grown rapidly in the past few years. Although originally marketed as an aid to assist habitual smokers wishing to quit tobacco smoking, consumers are increasingly viewing smoking substitute systems as desirable lifestyle accessories.

There are a number of different categories of smoking substitute systems, each utilizing a different smoking substitute approach.

One approach for a smoking substitute system is the so-called Heated Tobacco (“HT”) approach in which tobacco (rather than an “e-liquid”) is heated or warmed to release vapor. HT is also known as “heat-not-burn” (“HNB”). The tobacco may be leaf tobacco or reconstituted tobacco. The vapor may contain nicotine and/or flavorings. In the HT approach the intention is that the tobacco is heated but not burned, i.e., the tobacco does not undergo combustion.

A typical HT smoking substitute system may include a device and a consumable. The consumable may include the tobacco material. The device and consumable may be configured to be physically coupled together. In use, heat may be imparted to the tobacco material by a heating element of the device, wherein airflow through the tobacco material causes components in the tobacco material to be released as vapor. A vapor may also be formed from a carrier in the tobacco material (this carrier may for example include propylene glycol and/or vegetable glycerin) and additionally volatile compounds released from the tobacco. The released vapor may be entrained in the airflow drawn through the tobacco.

As the vapor passes through the consumable (entrained in the airflow) from the location of vaporization to an outlet of the consumable (e.g., a mouthpiece), the vapor cools and condenses to form an aerosol for inhalation by the user. The aerosol will normally contain the volatile compounds.

In HT smoking substitute systems, heating as opposed to burning the tobacco material is believed to cause fewer, or smaller quantities, of the more harmful compounds ordinarily produced during smoking. Consequently, the HT approach may reduce the odor and/or health risks that can arise through the burning, combustion, and pyrolytic degradation of tobacco.

There may be a need for improved design of smoking substitute systems, in particular HT smoking substitute systems, to enhance the user experience and improve the function of the HT smoking substitute system.

The present disclosure has been devised in the light of the above considerations.

### SUMMARY OF THE INVENTION

At its most general, the present invention relates to a smoking substitute device which is configured to detect an error event and an event of user misuse and alert the user of the same.

According to a first aspect of the present invention, there is provided a smoking substitute device comprising a controller and an output device. The controller is configured to detect an error event of the device and an event of user misuse of the device, whereby upon detecting an occurrence of said error event or event of user misuse, the controller is configured to alert the user of said error event or event of user misuse via the output device.

By providing a device comprising a controller that is configured to detect an error event and an event of user misuse of the device, a versatile device is provided which is able to monitor a range of adverse events which may be detrimental to normal device operation and alert the user so that they can take necessary corrective steps. Since the device monitors both device error events and user misuse it is able to inform the user when either of these occurs but also may distinguish between them so that a user is better informed of the root cause of a device malfunction so that appropriate corrective action may be taken.

Optional features will now be set out. These are applicable singly or in any combination with any aspect.

Optionally, the event of user misuse the event of user misuse comprises one or more of: (a) physical tampering with a cap of the device; (b) tampering with a user interface of the device; and (c) inappropriate selection of a mode of the device. In some embodiments the device comprises a cap which provides access to internal components of the device, such as a heater. Tampering with this cap (e.g., moving, lifting, or removing the cap) during device operation may risk damaging the device and may be dangerous for the user, so alerting the user to this provides a valuable device function. Similarly, it is beneficial for the user to be alerted to tampering with a user interface (e.g., pressing of buttons on the user interface in an unauthorized or unrecognized manner). Further, alerting the user to an inappropriate mode selection allows them to change to an appropriate mode, for example where the mode selection was made inadvertently.

In some embodiments, the event of user misuse comprises at least one of: (a) lifting a cap of the device when the device is in use; (b) one or more attempts to unlock the device with an incorrect user input, as determined by the controller by comparing the user input with a predetermined unlock pattern; and (c) an attempt to switch the device into a high temperature mode when the ambient temperature is determined by the controller to be above a predetermined threshold temperature. In some embodiments, if the controller detects that any of said events have occurred in may consider it as user misuse. For example, if the user lifts the cap of the device when the device is in operational mode, which may be dangerous for the user, the controller detects it as user misuse. In another example, if the repeated attempts to unlock the device with the incorrect sequence of button presses is made, which may be because of device being operated by anyone other than the rightful user (e.g., a child), the controller may conclude this as user misuse. Further in another example, if an attempt is made to switch the device into a high temperature mode (i.e., increase the power supply to the device heater to increase the level of heat provided by the heater) when the ambient temperature is detected to be above a predetermined threshold temperature, which may lead to overheating of the device, the controller may register this as user misuse. The controller may further be configured to alert the user when one or more of any of said events occur.

In some embodiments, the error event comprises at least one of: (a) electrical short-circuit; (b) electrical open-circuit; (c) microcontroller error; (d) overheating of one or more components of the device; (e) a detection of ambient temperature determined by the controller to be above a predetermined threshold temperature; (f) sensor error; and (g) user interface error. In some embodiments, overheating is determined based on a predetermined threshold temperature. The controller may be configured to detect an overheating error when a temperature sensor detects that the temperature within the device (e.g., adjacent the rod heater) exceeds a predetermined threshold. The skilled person is aware of suitable sensors and components which may be used to detect the above scenarios and pass information to the controller.

In some embodiments, the error event may comprise the following events (i) short circuit on heater rod, (ii) open circuit detection, (iii) microcontroller error, (iv) battery overheat, (v) heater overheats, (vi) ambient temperature too high or too low, (vii) puff sensor error and (viii) user interface error. A more versatile device is provided which can react intelligently to different scenarios. For example, the device is intelligent enough to detect one or more of the above listed error events and alert the user of such error or misuse.

In some embodiments, the controller is coupled to a heater and is configured to disable the heater in response to detecting an occurrence of one or more error events or one or more events of user misuse. In some embodiments, the device intelligently monitors all the activities performed on the device and intelligently reacts to these activities. For example, if the controller of the device detects that one or more of above error events or user misuse events has occurred, it may immediately disable the heater which prevents any harm being caused to the device or to the user.

Optionally, the output device is configured to generate one or more of visual, audio, and haptic feedback to alert the user to the error event or the event of user misuse. The output device may be selected from one or more of (i) one or more light sources, e.g., LEDs, (ii) one or more haptic feedback devices, (iii) one or more audio feedback devices, e.g., speakers. Said output means may be used by the output device to alert the user of the error event or event of user misuse. Thus, the device keeps the user well informed if any of the error event or event of user misuse occurs. In some embodiments, the output device comprises one or more LEDs. In some embodiments, the output device comprises one or more color change LEDs. In some embodiments, the output device is configured to generate visual feedback comprising a change in color of one or more LEDs for a predetermined period of time to alert the user to the error event or the event of user misuse. In some embodiments, the output device is configured to generate visual feedback comprising flashing of one or more LEDs for a predetermined period of time to alert the user to the error event or the event of user misuse. In some embodiments, the output device comprises a haptic feedback device, for example a vibrating device. In some embodiments, the output device is configured to vibrate for a predetermined period of time to alert the user to the error event or the event of user misuse. The feedback may be provided for a predetermined period of time (e.g., programmed within the device or chosen by the user), or until the error is determined to be rectified. In some embodiments, the output device is configured to generate two or more of (a) visual feedback comprising a change in color of one or more LEDs, (b) visual feedback comprising flashing of one or more LEDs, and (c) vibration, for a predetermined period of time, to alert the user to the error event or the event of user misuse. The two or more outputs may be generated simultaneously or sequentially.

For example, an LED on the user interface of the device may be lit up during normal use of the device in a first color (such as green) but will change to a second color (such as red) if the device detects one or more error events or events of user misuse. In another embodiment, an LED on the user interface of the device may be unlit during normal use of the device and may light up if the device detects one or more error events or events of user misuse. Alternatively, a first LED may be lit up during normal use of the device, and if the device detects one or more error events or events of user misuse a second LED will light up, wherein the first LED optionally extinguishes when the device detects the one or more error events or events of user misuse, and the first and second LEDs are optionally differently colored. In some embodiments, the LED output is steady. In some embodiments, the LED output is flashing. Optionally, simultaneously with the above LED outputs, the device may vibrate to provide haptic feedback when the device detects one or more error events or events of user misuse.

Such device output provides a clear indication to the user than an error or misuse has occurred.

In some embodiments, the controller is coupled to a memory configured to store data generated by the error event or event of user misuse. In this way the device is able to record instances of error or user misuse which may be used later, for example to identify any problems with the functioning of the device and troubleshoot them.

In some embodiments, the device is connectable to a further device to which the data generated by the error event or event of user misuse is transferable. For example, the device may be connectable, by wired connection or wirelessly, to a computer, the computer including software which is able to interpret the data to provide useful advice to the user.

In some embodiments, the output device is configured to provide distinct alerts for each of the error event and the event of user misuse. For example, the output device may be configured to provide a first type of alert for an error event and a second type of alert for an event of user misuse. This provides a clear indication to the user as to whether the error event has occurred or event of user misuse has occurred. Based on said indication the user may then take a decision whether he can fix the device himself or he needs to take the device to a technician for more extensive repair. In some embodiments, the output device is configured to provide distinct alerts for each of two or more error events. For example, the output device may be configured to provide a first alert for a first error event (such as a short-circuit) and a second alert for a second error event (such as overheating of the heater), wherein the first and second alerts are different. For example, the first alert may be a visual alert and the second alert may be a haptic alert. Similarly in some embodiments, the output device is configured to provide distinct alerts for each of two or more events of user misuse.

The device may comprise an elongate body. An end of the elongate body may be configured for engagement with an aerosol-forming article. For example, the body may be configured for engagement with a heated tobacco (HT) consumable (or heat-not-burn (HNB) consumable). The terms “heated tobacco” and “heat-not-burn” are used interchangeably herein to describe a consumable that is of the type that is heated rather than combusted (or are used interchangeably to describe a device for use with such a consumable). The device may comprise a cavity that is configured for receipt of at least a portion of the consumable (i.e., for engagement with the consumable). The aerosol-forming article may be of the type that comprises an aerosol former (e.g., carried by an aerosol-forming substrate).

The device may comprise a heater for heating the aerosol-forming article. The heater may comprise a heating element, which may be in the form of a rod that extends from the body of the device. The heating element may extend from the end of the body that is configured for engagement with the aerosol-forming article. In an embodiment, the heater of the device is configured to be disabled in response to the controller detecting an occurrence of one or more error events or one or more events of user misuse of the device.

The heater (and thus the heating element) may be rigidly mounted to the body. The heating element may be elongate so as to define a longitudinal axis and may, for example, have a transverse profile (i.e., transverse to a longitudinal axis of the heating element) that is substantially circular (i.e., the heating element may be generally cylindrical). Alternatively, the heating element may have a transverse profile that is rectangular (i.e., the heater may be a “blade heater”). The heating element may alternatively be in the shape of a tube (i.e., the heater may be a “tube heater”). The heating element may take other forms (e.g., the heating element may have an elliptical transverse profile). The shape and/or size (e.g., diameter) of the transverse profile of the heating element may be generally consistent for the entire length (or substantially the entire length) of the heating element.

The heating element may be between 15 mm and 25 mm long, e.g., between 18 mm and 20 mm long, e.g., around 19 mm long. The heating element may have a diameter of between 1.5 mm and 2.5 mm, e.g., a diameter between 2 mm and 2.3 mm, e.g., a diameter of around 2.15 mm.

The heating element may be formed of ceramic. The heating element may comprise a core (e.g., a ceramic core) comprising Al₂O₃. The core of the heating element may have a diameter of 1.8 mm to 2.1 mm, e.g., between 1.9 mm and 2 mm. The heating element may comprise an outer layer (e.g., an outer ceramic layer) comprising Al₂O₃. The thickness of the outer layer may be between 160 μm and 220 μm, e.g., between 170 μm and 190 μm, e.g., around 180 μm. The heating element may comprise a heating track, which may extend longitudinally along the heating element. The heating track may be sandwiched between the outer layer and the core of the heating element. The heating track may comprise tungsten and/or rhenium. The heating track may have a thickness of around 20 μm.

The heating element may be located in the cavity (of the device), and may extend (e.g., along a longitudinal axis) from an internal base of the cavity towards an opening of the cavity. The length of the heating element (i.e., along the longitudinal axis of the heater) may be less than the depth of the cavity. Hence, the heating element may extend for only a portion of the length of the cavity. That is, the heating element may not extend through (or beyond) the opening of the cavity.

The heating element may be configured for insertion into an aerosol-forming article (e.g., a HT consumable) when an aerosol-forming article is received in the cavity. In that respect, a distal end (i.e., distal from a base of the heating element where it is mounted to the device) of the heating element may comprise a tapered portion, which may facilitate insertion of the heating element into the aerosol-forming article. The heating element may fully penetrate an aerosol-forming article when the aerosol-forming article is received in the cavity. That is, the entire length, or substantially the entire length, of the heating element may be received in the aerosol-forming article.

The heating element may have a length that is less than, or substantially the same as, an axial length of an aerosol-forming substrate forming part of an aerosol-forming article (e.g., a HT consumable). Thus, when such an aerosol-forming article is engaged with the device, the heating element may only penetrate the aerosol-forming substrate, rather than other components of the aerosol-forming article. The heating element may penetrate the aerosol-forming substrate for substantially the entire axial length of the aerosol forming-substrate of the aerosol-forming article. Thus, heat may be transferred from (e.g., an outer circumferential surface of) the heating element to the surrounding aerosol-forming substrate, when penetrated by the heating element. That is, heat may be transferred radially outwardly (in the case of a cylindrical heating element) or, e.g., radially inwardly (in the case of a tube heater).

Where the heater is a tube heater, the heating element of the tube heater may surround at least a portion of the cavity. When the portion of the aerosol-forming article is received in the cavity, the heating element may surround a portion of the aerosol-forming article (i.e., so as to heat that portion of the aerosol-forming article). In particular, the heating element may surround an aerosol forming substrate of the aerosol-forming article. That is, when an aerosol-forming article is engaged with the device, the aerosol forming substrate of the aerosol-forming article may be located adjacent an inner surface of the (tubular) heating element. When the heating element is activated, heat may be transferred radially inwardly from the inner surface of the heating element to heat the aerosol forming substrate.

The cavity may comprise a (e.g., circumferential) wall (or walls) and the (tubular) heating element may extend around at least a portion of the wall(s). In this way, the wall may be located between the inner surface of the heating element and an outer surface of the aerosol-forming article. The wall (or walls) of the cavity may be formed from a thermally conductive material (e.g., a metal) to allow heat conduction from the heating element to the aerosol-forming article. Thus, heat may be conducted from the heating element, through the cavity wall (or walls), to the aerosol-forming substrate of an aerosol-forming article received in the cavity.

In some embodiments the device may comprise a cap disposed at the end of the body that is configured for engagement with an aerosol-forming article. Where the device comprises a heater having a heating element, the cap may at least partially enclose the heating element. The cap may be moveable between an open position in which access is provided to the heating element, and a closed position in which the cap at least partially encloses the heating element. The cap may be slidably engaged with the body of the device and may be slidable between the open and closed positions.

The cap may define at least a portion of the cavity of the device. That is, the cavity may be fully defined by the cap, or each of the cap and body may define a portion of the cavity. Where the cap fully defines the cavity, the cap may comprise an aperture for receipt of the heating element into the cavity (when the cap is in the closed position). The cap may comprise an opening to the cavity. The opening may be configured for receipt of at least a portion of an aerosol-forming article. That is, an aerosol-forming article may be inserted through the opening and into the cavity (so as to be engaged with the device).

The cap may be configured such that when an aerosol-forming article is engaged with the device (e.g., received in the cavity), only a portion of the aerosol-forming article is received in the cavity. That is, a portion of the aerosol-forming article (not received in the cavity) may protrude from (i.e., extend beyond) the opening. This (protruding) portion of the aerosol-forming article may be a terminal (e.g., mouth) end of the aerosol-forming article, which may be received in a user's mouth for the purpose of inhaling aerosol formed by the device.

The device may comprise a power source or may be connectable to a power source (e.g., a power source separate to the device). The power source may be electrically connectable to the heater. In that respect, altering (e.g., toggling) the electrical connection of the power source to the heater may affect a state of the heater. For example, toggling the electrical connection of the power source to the heater may toggle the heater between an on state and an off state. The power source may be a power store. For example, the power source may be a battery or rechargeable battery (e.g., a lithium-ion battery).

The device may comprise an input connection (e.g., a USB port, Micro USB port, USB-C port, etc.). The input connection may be configured for connection to an external source of electrical power, such as a mains electrical supply outlet. The input connection may, in some cases, be used as a substitute for an internal power source (e.g., battery or rechargeable battery). That is, the input connection may be electrically connectable to the heater (for providing power to the heater). Hence, in some forms, the input connection may form at least part of the power source of the device.

Where the power source comprises a rechargeable power source (such as a rechargeable battery), the input connection may be used to charge and recharge the power source.

The device may comprise a user interface (UI). In some embodiments the UI may include input means to receive operative commands from the user. The input means of the Ul may allow the user to control at least one aspect of the operation of the device. In some embodiments the input means may comprise a power button to switch the device between an on state and an off state.

In some embodiments the UI may additionally or alternatively comprise output means to convey information to the user. In some embodiments the output means may also comprise a light to indicate a condition of the device (and/or the aerosol-forming article) to the user. The condition of the device (and/or aerosol-forming article) indicated to the user may comprise a condition indicative of the operation of the heater. For example, the condition may comprise whether the heater is in an off state or an on state. In some embodiments, the Ul unit may comprise at least one of a button, a display, a touchscreen, a switch, a light, and the like. For example, the output means may comprise one or more (e.g., two, three, four, etc.) light-emitting diodes (“LEDs”) that may be located on the body of the device.

The device may further comprise a puff sensor (e.g., airflow sensor), which form part of the input means of the UI. The puff sensor may be configured to detect a user drawing on an end (i.e., a terminal (mouth) end) of the aerosol-forming article. The puff sensor may, for example, be a pressure sensor or a microphone. The puff sensor may be configured to produce a signal indicative of a puff state. The signal may be indicative of the user drawing (an aerosol from the aerosol-forming article) such that it is, e.g., in the form of a binary signal. Alternatively or additionally, the signal may be indicative of a characteristic of the draw (e.g., a flow rate of the draw, length of time of the draw, etc.).

The device may comprise a controller or may be connectable to a controller that may be configured to control at least one function of the device. The controller may comprise a microcontroller that may, e.g., be mounted on a printed circuit board (PCB). The controller may also comprise a memory, e.g., non-volatile memory. The memory may include instructions, which, when implemented, may cause the controller to perform certain tasks or steps of a method. Where the device comprises an input connection, the controller may be connected to the input connection.

The controller may be coupled to the heater and configured to control the operation of the heater (and, e.g., the heating element). The controller is configured to detect an error event and an event of user misuse of the device and provide an alert to the user of said error event or event of user misuse via an output device. Thus the controller is capable of detecting both an error event and an event of user misuse, providing more useful monitoring of device function. Further, the controller may be configured to control vaporization of an aerosol forming part of an aerosol-forming article engaged with the device. The controller may be configured to control the voltage applied by power source to the heater. For example, the controller may be configured to toggle between applying a full output voltage (of the power source) to the heater and applying no voltage to the heater. Alternatively or additionally, the control unit may implement a more complex heater control protocol.

The device may further comprise a voltage regulator to regulate the output voltage supplied by the power source to form a regulated voltage. The regulated voltage may subsequently be applied to the heater. In some embodiment, the voltage regulator may be used to control the supply of voltage to the heater when an error event or an event of user misuse is detected. For example, when an error event or an event of user misuse is detected, the voltage may be reduced to reduce the power supplied to the heater, in some embodiments to zero, thereby disabling the heater.

In some embodiments, where the device comprises a UI, the controller may be operatively connected to one or more components of the UI. The controller may be configured to receive command signals from an input means of the UI. The controller may be configured to control the heater in response to the command signals. For example, the controller may be configured to receive “on” and “off” command signals from the UI and, in response, may control the heater so as to be in a corresponding on or off state.

The controller may be configured to send output signals to a component of the UI. The UI may be configured to convey information to a user, via an output means, in response to such output signals (received from the controller). For example, where the device comprises one or more LEDs, the LEDs may be operatively connected to the controller. Hence, the controller may be configured to control the illumination of the LEDs (e.g., in response to an output signal). For example, the controller may be configured to control the illumination of the LEDs according to (e.g., an on or off) state of the heater.

Where the device comprises a sensor (e.g., a puff/airflow sensor), the controller may be operatively connected to the sensor. The controller may be configured to receive a signal from the sensor (e.g., indicative of a condition of the device and/or engaged aerosol-forming article). The controller may be configured to control the heater, or an aspect of the output means, based on the signal from the sensor.

The device may comprise a wireless interface configured to communicate wirelessly (e.g., via Bluetooth (e.g., a Bluetooth low-energy connection) or Wi-Fi) with an external device. Similarly, the input connection may be configured for wired connection to an external device so as to provide communication between the device and the external device.

The external device may be a mobile device. For example, the external device may be a smart phone, tablet, smart watch, or smart car. An application (e.g., app) may be installed on the external device (e.g., mobile device). The application may facilitate communication between the device and the external device via the wired or wireless connection.

The wireless or wired interface may be configured to transfer signals between the external device and the controller of the device. In this respect, the controller may control an aspect of the device in response to a signal received from an external device.

Alternatively or additionally, an external device may respond to a signal received from the device (e.g., from the controller of the device).

In a second aspect, there is provided a system (e.g., a smoking substitute system) comprising a device according to the first aspect and an aerosol-forming article. The aerosol-forming article may comprise an aerosol-forming substrate at an upstream end of the aerosol-forming article. The article may be in the form of a smoking substitute article, e.g., heated tobacco (HT) consumable (also known as a heat-not-burn (HNB) consumable).

As used herein, the terms “upstream” and “downstream” are intended to refer to the flow direction of the vapor/aerosol, i.e., with the downstream end of the article/consumable being the mouth end or outlet where the aerosol exits the consumable for inhalation by the user. The upstream end of the article/consumable is the opposing end to the downstream end.

The aerosol-forming substrate is capable of being heated to release at least one volatile compound that can form an aerosol. The aerosol-forming substrate may be located at the upstream end of the article/consumable.

In order to generate an aerosol, the aerosol-forming substrate comprises at least one volatile compound that is intended to be vaporized/aerosolized and that may provide the user with a recreational and/or medicinal effect when inhaled. Suitable chemical and/or physiologically active volatile compounds include the group consisting of nicotine, cocaine, caffeine, opiates and opioids, cathine and cathinone, kavalactones, mysticin, beta-carboline alkaloids, salvinorin A together with any combinations, functional equivalents to, and/or synthetic alternatives of the foregoing.

The aerosol-forming substrate may comprise plant material. The plant material may comprise least one plant material selected from the list including Amaranthus dubius, Arctostaphylos uva-ursi (Bearberry), Argemone mexicana, Amica, Artemisia vulgaris, Yellow Tees, Galea zacatechichi, Canavalia maritima (Baybean), Cecropia mexicana (Guamura), Cestrum noctumum, Cynoglossum virginianum (wild comfrey), Cytisus scoparius, Damiana, Entada rheedii, Eschscholzia califomica (California Poppy), Fittonia albivenis, Hippobroma longiflora, Humulus japonica (Japanese Hops), Humulus lupulus (Hops), Lactuca virosa (Lettuce Opium), Laggera alata, Leonotis leonurus, Leonurus cardiaca (Motherwort), Leonurus sibiricus (Honeyweed), Lobelia cardinalis, Lobelia inflata (Indian-tobacco), Lobelia siphilitica, Nepeta cataria (Catnip), Nicotiana species (Tobacco), Nymphaea alba (White Lily), Nymphaea caerulea (Blue Lily), Opium poppy, Passiflora incamata (Passionflower), Pedicularis densiflora (Indian Warrior), Pedicularis groenlandica (Elephant's Head), Salvia divinorum, Salvia dorrii (Tobacco Sage), Salvia species (Sage), Scutellaria galericulata, Scutellaria lateriflora, Scutellaria nana, Scutellaria species (Skullcap), Sida acuta (Wireweed), Sida rhombifolia, Silene capensis, Syzygium aromaticum (Clove), Tagetes lucida (Mexican Tarragon), Tarchonanthus camphoratus, Tumera diffusa (Damiana), Verbascum (Mullein), Zamia latifolia (Maconha Brava) together with any combinations, functional equivalents to, and/or synthetic alternatives of the foregoing.

The plant material may be tobacco. Any type of tobacco may be used. This includes, but is not limited to, flue-cured tobacco, burley tobacco, Maryland Tobacco, dark-air cured tobacco, oriental tobacco, dark-fired tobacco, perique tobacco and rustica tobacco. This also includes blends of the above-mentioned tobaccos.

The tobacco may comprise one or more of leaf tobacco, stem tobacco, tobacco powder, tobacco dust, tobacco derivatives, expanded tobacco, homogenized tobacco, shredded tobacco, extruded tobacco, cut rag tobacco and/or reconstituted tobacco (e.g., slurry recon or paper recon).

The aerosol-forming substrate may comprise a gathered sheet of homogenized (e.g., paper/slurry recon) tobacco or gathered shreds/strips formed from such a sheet.

The aerosol-forming substrate may comprise one or more additives selected from humectants, flavorants, fillers, and aqueous/non-aqueous solvents and binders.

The flavorant may be provided in solid or liquid form. It may include menthol, licorice, chocolate, fruit flavor (including, e.g., citrus, cherry etc.), vanilla, spice (e.g., ginger, cinnamon) and tobacco flavor. The flavorant may be evenly dispersed throughout the aerosol-forming substrate or may be provided in isolated locations and/or varying concentrations throughout the aerosol-forming substrate.

The aerosol-forming substrate may be formed in a substantially cylindrical shape such that the article/consumable resembles a conventional cigarette. It may have a diameter of between 5 and 10 mm, e.g., between 6 and 9 mm or 6 and 8 mm, e.g., around 7 mm. It may have an axial length of between 10 and 15 mm, e.g., between 11 and 14 mm such as around 12 or 13 mm.

The article/consumable may comprise at least one filter element. There may be a terminal filter element at the downstream/mouth end of the article/consumable.

The or at least one of the filter element(s) (e.g., the terminal filter element) may be comprised of cellulose acetate or polypropylene tow. The at least one filter element (e.g., the terminal filter element) may be comprised of activated charcoal. The at least one filter element (e.g., the terminal element) may be comprised of paper. The or each filter element may be at least partly (e.g., entirely) circumscribed with a plug wrap, e.g., a paper plug wrap.

The terminal filter element (at the downstream end of the article/consumable) may be joined to the upstream elements forming the article/consumable by a circumscribing tipping layer, e.g., a tipping paper layer. The tipping paper may have an axial length longer than the axial length of the terminal filter element such that the tipping paper completely circumscribes the terminal filter element plus the wrapping layer surrounding any adjacent upstream element.

In some embodiments, the article/consumable may comprise an aerosol-cooling element which is adapted to cool the aerosol generated from the aerosol-forming substrate (by heat exchange) before being inhaled by the user.

The article/consumable may comprise a spacer element that defines a space or cavity between the aerosol-forming substrate and the downstream end of the consumable. The spacer element may comprise a cardboard tube. The spacer element may be circumscribed by the (paper) wrapping layer.

According to a third aspect of the present invention, there is provided a method of using the system according to the second aspect, the method comprising inserting the consumable into the device; and heating the article using the heater of the device.

In some embodiments the method may comprise inserting the article into a cavity within a body of the device and penetrating the article with the heating element of the device upon insertion of the article.

According to a fourth aspect of the present invention, there is provided a method of operating a smoking substitute device, said smoking substitute device being configured to detect an error event of the device and an event of user misuse of the device, the method comprising: detecting an occurrence of an error event or an event of user misuse; and alerting the user to said error event or event of user misuse.

In some embodiments, the event of user misuse comprises one or more of: (a) physical tampering with a cap of the device; (b) tampering with a user interface of the device; and (c) inappropriate selection of a mode of the device. In some embodiments, the event of user misuse comprises at least one of: (a) lifting a cap of the device when the device is in use; (b) one or more attempts to unlock the device with an incorrect user input, as determined by the controller by comparing the user input with a predetermined unlock pattern; and (c) an attempt to switch the device into a high temperature mode when the ambient temperature is determined by the controller to be above a predetermined threshold temperature.

In some embodiments, the error event comprises at least one of: (a) electrical short-circuit; (b) electrical open-circuit; (c) microcontroller error; (d) overheating of one or more components of the device; (e) a detection of ambient temperature determined by the controller to be above a predetermined threshold temperature; (f) sensor error; and (g) user interface error. In some embodiments, the error event comprises at least one of: short circuit on a heater, open circuit detection, microcontroller error, battery overheat, overheating of a heater, ambient temperature too high or too low, puff sensor error and user interface error.

In some embodiments, the method further comprises disabling the heater of the device when the occurrence of an error event or an event of user misuse is detected.

In some embodiments, alerting comprises generating one of visual, audio, or haptic feedback to alert the user of the error event or event of user misuse.

The invention includes the combination of the aspects and preferred features described except where such a combination is clearly impermissible or expressly avoided.

The skilled person will appreciate that except where mutually exclusive, a feature or parameter described in relation to any one of the above aspects may be applied to any other aspect. Furthermore, except where mutually exclusive, any feature or parameter described herein may be applied to any aspect and/or combined with any other feature or parameter described herein.

### SUMMARY OF THE FIGURES

So that the invention may be understood, and so that further aspects and features thereof may be appreciated, embodiments illustrating the principles of the invention will now be discussed in further detail with reference to the accompanying figures, in which:

FIG. 1A is a schematic of a smoking substitute system;

FIG. 1B is a schematic of a variation of the smoking substitute system of FIG. 1A;

FIG. 2A is a front view of a first embodiment of the smoking substitute system with the consumable engaged with the device;

FIG. 2B is a front view of the first embodiment of the smoking substitute system with the consumable disengaged from the device;

FIG. 2C is a section view of the consumable of the first embodiment of-the smoking substitute system;

FIG. 2D is a detailed view of an end of the device of the first embodiment of the smoking substitute system;

FIG. 2E is a section view of the first embodiment of the substitute smoking system;

FIG. 3 is a flowchart illustrating method for detecting an occurrence of an error event or an event of user misuse.

### DETAILED DESCRIPTION OF THE INVENTION

Aspects and embodiments of the present invention will now be discussed with reference to the accompanying figures. Further aspects and embodiments will be apparent to those skilled in the art. All documents mentioned in this text are incorporated herein by reference.

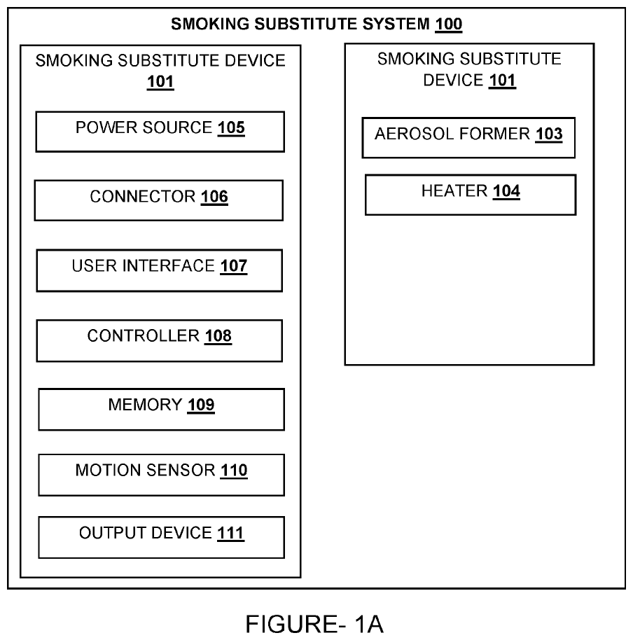

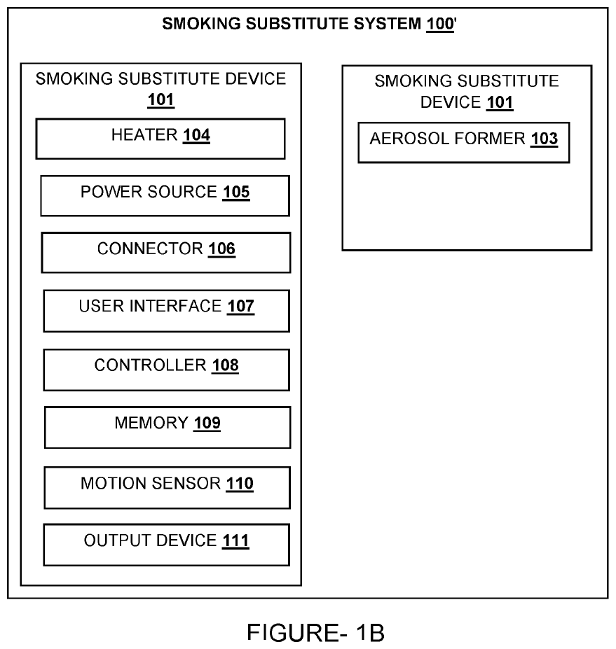

FIG. 1A is a schematic providing a general overview of a smoking substitute system 100. The system 100 includes a substitute smoking device 101 and an aerosol-forming article in the form of a consumable 102, which comprises an aerosol former 103. The system is configured to vaporize the aerosol former by heating the aerosol former 103 (so as to form a vapor/aerosol for inhalation by a user).

In the illustrated system, the heater 104 forms part of the consumable 102 and is configured to heat the aerosol former 103. Heat from the heater 104 vaporizes the aerosol former 103 to produce a vapor. The vapor subsequently condenses to form an aerosol, which is ultimately inhaled by the user. In some embodiments, the heater 104 is configured to be deactivated/disabled in the event of an error or user misuse being detected.

The system 100 further comprises a power source 105 that forms part of the device 101. In other embodiments the power source 105 may be external to (but connectable to) the device 101. The power source 105 is electrically connectable to the heater 104 such that the power source 105 is able to supply power to the heater 104 (i.e., for the purpose of heating the aerosol former 103). Thus, control of the electrical connection of the power source 105 to the heater 104 provides control of the state of the heater 104. The power source 105 may be a power store, for example a battery or rechargeable battery (e.g., a lithium-ion battery).

The system 100 further comprises an I/O module comprising a connector 106 (e.g., in the form of a USB port, Micro USB port, USB-C port, etc.). The connector 106 is configured for connection to an external source of electrical power, e.g., a mains electrical supply outlet. The connector 106 may be used in substitution for the power source 105. That is the connector 106 may be electrically connectable to the heater 104 so as to supply electricity to the heater 104. In such embodiments, the device may not include a power source, and the power source of the system may instead comprise the connector 106 and an external source of electrical power (to which the connector 106 provides electrical connection).

In some embodiments, the connector 106 may be used to charge and recharge the power source 105 where the power source 105 includes a rechargeable battery.

The system 100 also comprises a user interface (UI) 107. Although not shown, the UI 107 may include input means to receive commands from a user. The input means of the UI 107 allows the user to control at least one aspect of the operation of the system 100. The input means may, for example, be in the form of a button, touchscreen, switch, microphone, etc.

The UI 107 also comprises output means to convey information to the user. The output means may, for example, comprise lights (e.g., LEDs), a display screen, speaker, vibration generator, etc.

The system 100 further comprises a controller 108 and a memory 109 operatively coupled to the controller 108 that is configured to control at least one function of the device 101. In the illustrated embodiment, the controller 108 is a component of the device 101, but in other embodiments may be separate from (but connectable to) the device 101. The controller 108 is configured to detect an error event and a user misuse event and alert the user about the occurrence of said event via an output device 111. The controller 108 is further configured to control the operation of the heater 104, for example the controller may be configured to disable the heater 104 when the error event or the user misuse event is detected. The controller 108 may be further configured to control operation of the heater 104, for example, may be configured to control the voltage applied from the power source 105 to the heater 104. The controller 108 may be configured to toggle the supply of power to the heater 104 between an on state, in which the full output voltage of the power source 105 is applied to the heater 104, and an off state, in which the no voltage is applied to the heater 104. Further, in an example, the controller 108 may be configured to disconnect the power supply to the heater 104 when at least one of the error events or the user misuse events is detected.

Although not shown, the system 100 may also comprise a voltage regulator to regulate the output voltage from the power source 105 to form a regulated voltage. The regulated voltage may then be applied to the heater 104.

In addition to being connected to the heater 104, the controller 108 is operatively connected to the UI 107. Thus, the controller 108 may receive an input signal from the input means of the UI 107. Similarly, the controller 108 may transmit output signals to the UI 107. In response, the output means of the UI 107 may convey information, based on the output signals, to a user.

Further, the system may also comprise a sensor 110 coupled with the controller 108 within the smoking substitute device 101. The sensor 110 may be a motion sensor, temperature sensor, puff sensor or any other similar sensor mounted inside the device and configured to generate an input in response to detection of one of the following events such as, lifting of cap in operating mode, attempt made to use the device at high temperature when the ambient temperature is detected too high, short circuit on heater rod, open circuit detection, microcontroller error, battery overheat, heater overheats, puff sensor error and user interface error and provide the said input to the controller 108. The controller 108 is further configured to provide an alert to the user in response to detection of the error events and the event of user misuse of the device 101.

FIG. 1B is a schematic showing a variation of the system 100 of FIG. 1A. In the system 100′ of FIG. 1B, the heater 104 forms part of the device 101, rather than the consumable 102. In this variation, the heater 104 is electrically connected to the power source 105.

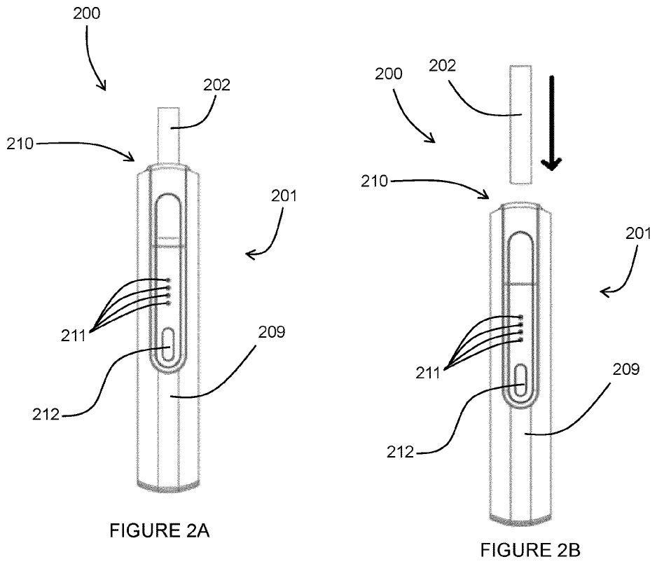

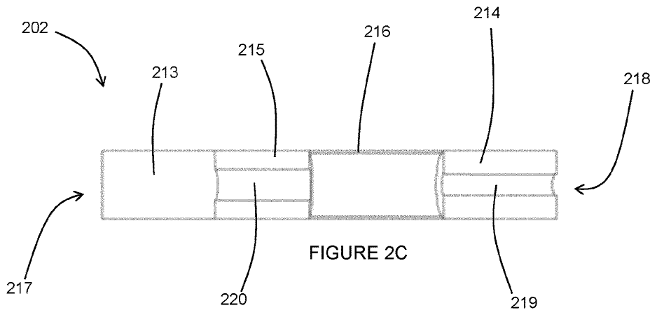

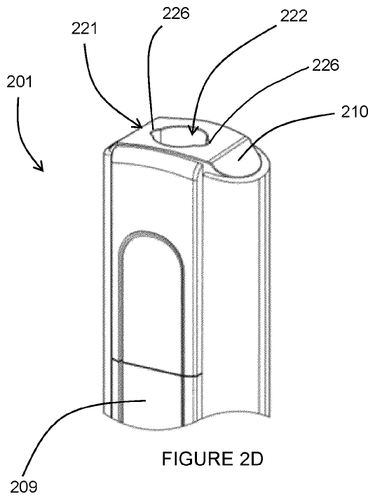

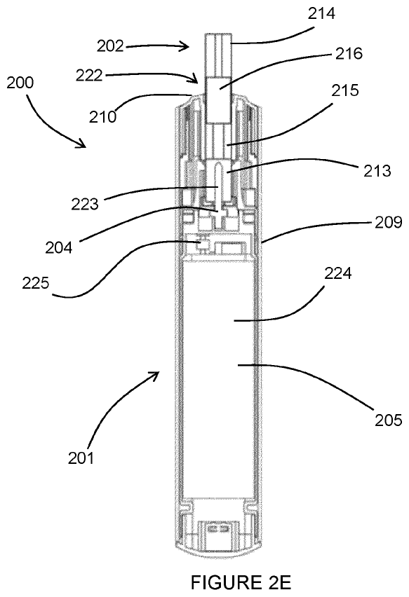

FIG. 2A and FIG. 2B illustrate a heated-tobacco (HT) smoking substitute system 200. The system 200 is an example of the systems 100, 100′ described in relation to FIG. 1A or FIG. 1B. System 200 includes an HT device 201 that is configured to detect an error event and an event of user misuse of the device and an HT consumable 202. The description of FIG. 1A and FIG. 1B above is applicable to the system 200 of FIG. 2A and FIG. 2B and will thus not be repeated.

The device 201 and the consumable 202 are configured such that the consumable 202 can be engaged with the device 201. FIG. 2A shows the device 201 and the consumable 202 in an engaged state, whilst FIG. 2B shows the device 201 and the consumable 202 in a disengaged state.

The device 201 comprises a body 209 and cap 210. In use the cap 210 is engaged at an end of the body 209. Although not apparent from the figures, the cap 210 is moveable relative to the body 209. In particular, the cap 210 is slidable and can slide along a longitudinal axis of the body 209.

The device 201 comprises an output means (forming part of the UI of the device 201) in the form of a plurality of light-emitting diodes (LEDs) 211 arranged linearly along the longitudinal axis of the device 201 and on an outer surface of the body 209 of the device 201. A button 212 is also arranged on an outer surface of the body 209 of the device 201 and is axially spaced (i.e., along the longitudinal axis) from the plurality of LEDs 211.

FIG. 2C show a detailed section view of the consumable 202 of the system 200. The consumable 202 generally resembles a cigarette. In that respect, the consumable 202 has a generally cylindrical form with a diameter of 7 mm and an axial length of 70 mm. The consumable 202 comprises an aerosol forming substrate 213, a terminal filter element 214, an upstream filter element 215 and a spacer element 216. In other embodiments, the consumable may further comprise a cooling element. A cooling element may exchange heat with vapor that is formed by the aerosol-forming substrate 213 in order to cool the vapor so as to facilitate condensation of the vapor.

The aerosol-forming substrate 213 is substantially cylindrical and is located at an upstream end 217 of the consumable 202 and comprises the aerosol former of the system 200. In that respect, the aerosol forming substrate 213 is configured to be heated by the device 201 to release a vapor. The released vapor is subsequently entrained in an airflow flowing through the aerosol-forming substrate 213. The airflow is produced by the action of the user drawing on a downstream end 218 (i.e., terminal or mouth end) of the consumable 202.

In the present embodiment, the aerosol forming substrate 213 comprises tobacco material that may, for example, include any suitable parts of the tobacco plant (e.g., leaves, stems, roots, bark, seeds, and flowers). The tobacco may comprise one or more of leaf tobacco, stem tobacco, tobacco powder, tobacco dust, tobacco derivatives, expanded tobacco, homogenized tobacco, shredded tobacco, extruded tobacco, cut rag tobacco and/or reconstituted tobacco (e.g., slurry recon or paper recon). For example, the aerosol-forming substrate 213 may comprise a gathered sheet of homogenized (e.g., paper/slurry recon) tobacco or gathered shreds/strips formed from such a sheet.

In order to generate an aerosol, the aerosol forming substrate 213 comprises at least one volatile compound that is intended to be vaporized/aerosolized and that may provide the user with a recreational and/or medicinal effect when inhaled. The aerosol-forming substrate 213 may further comprise one or more additives. For example, such additives may be in the form of humectants (e.g., propylene glycol and/or vegetable glycerin), flavorants, fillers, aqueous/non-aqueous solvents and/or binders.

The terminal filter element 214 is also substantially cylindrical and is located downstream of the aerosol forming substrate 213 at the downstream end 218 of the consumable 202. The terminal filter element 214 is in the form of a hollow bore filter element having a bore 219 (e.g., for airflow) formed therethrough. The diameter of the bore 219 is 2 mm. The terminal filter element 214 is formed of a porous (e.g., monoacetate) filter material. As set forth above, the downstream end 218 of the consumable 202 (i.e., where the terminal filter 214 is located) forms a mouthpiece portion of the consumable 202 upon which the user draws. Airflow is drawn from the upstream end 217, thorough the components of the consumable 202, and out of the downstream end 218. The airflow is driven by the user drawing on the downstream end 218 (i.e., the mouthpiece portion) of the consumable 202.

The upstream filter element 215 is located axially adjacent to the aerosol-forming substrate 213, between the aerosol-forming substrate 213 and the terminal filter element 214. Like the terminal filter 214, the upstream filter element 215 is in the form of a hollow bore filter element, such that it has a bore 220 extending axially therethrough. In this way, the upstream filter 215 may act as an airflow restrictor. The upstream filter element 215 is formed of a porous (e.g., monoacetate) filter material. The bore 220 of the upstream filter element 215 has a larger diameter (3 mm) than the terminal filter element 214.

The spacer 216 is in the form of a cardboard tube, which defines a cavity or chamber between the upstream filter element 215 and the terminal filter element 214. The spacer 216 acts to allow both cooling and mixing of the vapor/aerosol from the aerosol-forming substrate 213. The spacer has an external diameter of 7 mm and an axial length of 14 mm.

Although not apparent from the figure, the aerosol-forming substrate 213, upstream filter 215 and spacer 216 are circumscribed by a paper wrapping layer. The terminal filter 214 is circumscribed by a tipping layer that also circumscribes a portion of the paper wrapping layer (so as to connect the terminal filter 214 to the remaining components of the consumable 202). The upstream filter 215 and terminal filter 214 are circumscribed by further wrapping layers in the form of plug wraps.

Returning now to the device 201, FIG. 2D illustrates a detailed view of the end of the device 201 that is configured to engage with the consumable 202. The cap 210 of the device 201 includes an opening 221 to an internal cavity 222 (more apparent from FIG. 2D) defined by the cap 210. The opening 221 and the cavity 222 are formed so as to receive at least a portion of the consumable 202. During engagement of the consumable 202 with the device 201, a portion of the consumable 202 is received through the opening 221 and into the cavity 222. After engagement (see FIG. 2B), the downstream end 218 of the consumable 202 protrudes from the opening 221 and thus also protrudes from the device 201. The opening 221 includes laterally disposed notches 226. When a consumable 202 is received in the opening 221, these notches 226 remain open and could, for example, be used for retaining a cover in order to cover the end of the device 201.

FIG. 2E shows a cross section through a central longitudinal plane through the device 201. The device 201 is shown with the consumable 202 engaged therewith.

The device 201 comprises a heater 204 comprising heating element 223. The heater 204 forms part of the body 209 of the device 201 and is rigidly mounted to the body 209. In the illustrated embodiment, the heater 204 is a rod heater with a heating element 223 having a circular transverse profile. In other embodiments the heater may be in the form of a blade heater (e.g., heating element with a rectangular transverse profile) or a tube heater (e.g., heating element with a tubular form).

The heating element 223 of the heater 204 projects from an internal base of the cavity 222 along a longitudinal axis towards the opening 221. As is apparent from the figure, the length (i.e., along the longitudinal axis) of the heating element is less than a depth of the cavity 222. In this way, the heating element 223 does not protrude from or extend beyond the opening 221.

When the consumable 202 is received in the cavity 222 (as is shown in FIG. 2E), the heating element 223 penetrates the aerosol-forming substrate 213 of the consumable 202. In particular, the heating element 223 extends for nearly the entire axial length of the aerosol-forming substrate 213 when inserted therein. Thus, when the heater 204 is activated, heat is transferred radially from an outer circumferential surface the heating element 223 to the aerosol-forming substrate 213.

The device 201 further comprises an electronics cavity 224. A power source, in the form of a rechargeable battery 205 (a lithium-ion battery), is located in electronics cavity 224.

The device 201 includes a connector (i.e., forming part of an IO module of the device 201) in the form of a USB port 206. The connector may alternatively be, for example, a micro-USB port or a USB-C port for examples. The USB port 206 may be used to recharge the rechargeable battery 205.

The device 201 includes a controller (not shown) located in the electronics cavity 224. The controller comprises a microcontroller mounted on a printed circuit board (PCB). The USB port 206 is also connected to the controller 208 (i.e., connected to the PCB and microcontroller). The controller 208 is configured to control at least one function of the device 201. For example, the controller 208 is configured to control the operation of the heater 204. Such control of the operation of the heater 204 may be accomplished by the controller toggling the electrical connection of the rechargeable battery 205 to the heater 204. For example, the controller 208 is configured to control the heater 204 in response to a user depressing the button 212. Depressing the button 212 may cause the controller to allow a voltage (from the rechargeable battery 205) to be applied to the heater 204 (so as to cause the heating element 223 to be heated).

In one embodiment, the controller 208 is configured to detect an event of user misuse of the device 201. For example, the controller 208 detects that the cap 210 of the device 201 has been lifted away from the body 209 of the device 201 during a smoking session when the device is operational. This is detected my means of a sensor (not shown) which detects the movement of the cap away from the body of the device. In an embodiment, this is a light sensor which detects the increase in ambient light level when the cap is opened. When the controller 208 detects this movement of the cap 210, the controller is configured to send a signal to one of the LEDs 211 to light up. In an embodiment, the LEDs 211 are color change LEDs, and when the controller 208 detects the movement of the cap 210, the controller is configured to send a signal to one of the LEDs 211 to light up in a particular color which differs from the usual color of the LED when indicating other device functions. In addition, the controller 208 is also configured to deactivate the heater 204 by preventing the supply of power to the heater 204 when the controller 208 detects that the cap 210 of the device 201 has been lifted away from the body 209 of the device 201.

In another embodiment, the controller 208 detects tampering with the user interface of the device.

If a press of the button 212 does not match a predetermined, stored unlock pattern (e.g., duration of press, or number of discrete presses), then the controller is configured to send a signal to one of the LEDs 211 to light up. In addition, the controller 208 is configured to simultaneously activate a haptic feedback device (not shown) to generate vibration of the device. Thus the user is informed that the device has not been unlocked because the unlock pattern did not match a predetermined pattern.

In another embodiment, the controller 208 detects the ambient temperature, for example by means of a temperature sensor (not shown) within the body 209 of the device. If this ambient temperature is detected to be above a predetermined, stored threshold temperature value, then the controller 208 is configured to send a signal to one of the LEDs 211 to light up when the user attempts to increase the power supply to the heater (e.g., by attempting to switch the device into a “high power” or “high temperature” mode). In an embodiment, the LED which lights up is also configured to flash. This indicates to the user that the high temperature mode is currently inaccessible to preserve an acceptably low ambient temperature within the device.

The detection of said events may be detected by the controller 208 in response to receiving inputs from the sensor 110, wherein the sensor 110 may be one of a temperature sensor, piezoelectric sensor, touch sensor, biometric sensor, or any other similar sensor.

In another embodiment, the controller 208 is configured to detect an error event of the device 201. In an exemplary embodiment, the controller 208 is configured to detect a short-circuit across the heater 204, e.g., through detection of a surge in current. When the controller 208 detects this short-circuit, the controller is configured to send a signal to one of the LEDs 211 to light up. The controller is simultaneously configured to cut power to the heater to protect sensitive electrical components within the device. In an embodiment, the LED remains lit until the short-circuit error has been rectified, for example by a technician. Thus the user is informed that an error has occurred which requires expert attention.

Similarly, in another embodiment the controller 208 is configured to detect an open circuit, e.g., caused by a broken wire. This may be detected by the controller through measurement of the resistance in the circuit. When the controller 208 detects this open circuit, the controller is configured to send a signal to one of the LEDs 211 to light up. In an embodiment, the LED remains lit until the open circuit error has been rectified, for example by a technician. Thus the user is informed that an error has occurred which requires expert attention.

In an exemplary embodiment, the controller 208 is configured to detect overheating of the power source 205 within the device 201. This is detected by a temperature sensor (not shown) located close to the power source. The controller is configured to detect overheating of the power source when the temperature measured by the sensor exceeds a predetermined, stored temperature value. When the controller 208 detects such overheating, the controller is configured to send a signal to one of the LEDs 211 to light up. The controller is simultaneously configured to cut power supplied by the power source to prevent further overheating and prevent dangerous events such as battery fire.

Said detection of an error event may be achieved by the controller 208 in response to receiving inputs from the sensor 110, wherein the sensor 110 may be one of a temperature sensor, piezoelectric sensor, puff sensor or any other similar sensor.

The controller 208 upon detecting occurrence of any of the said error event or any of the said user misuse event, is configured to provide an alert to the user indicating that an error or misuse event has occurred in the device 201. Precisely, the controller 208 is configured to provide the alert signal to the output device 111. The output device 111 may be configured to generate one of visual, audio, and haptic feedbacks to alert the user of the error event or event of user misuse. To achieve this the output device 111 may include a plurality of LEDs, audio sensor and haptic sensor embedded therein. In an embodiment, the output device 111 comprises the LEDs 211 on the user interface. In an embodiment, the output device 111 further comprises an internal haptic feedback device (not shown) which generates vibration. In an embodiment, the output device 111 comprises a speaker (not shown).

The controller 208 may be further configured to disable the heater 204 of the device 201 upon detecting that one of error event and/or user misuse event has taken place. By disabling heater 204 the controller 208 ensures that the device 201 may not be able to operate any further as the same can cause damage to the device or to the user.

In an embodiment, the output device 111 is configured to provide distinct alerts for each of an error event and a user misuse event. In one example, the controller 208 is configured, via the output device 111, to generate a first alert comprising a steady LED 211 when the controller 208 detects a user misuse, whereas the controller 208 is configured to generate a second alert comprising a flashing LED 211 when the controller 208 detects a device error event. Thus the user is informed of the type of event by the type of alert generated and is able to take appropriate action.

The controller 208 is also configured to control the LEDs 211 in response to (e.g., a detected) a condition of the device 201 or the consumable 202. For example, the controller may control the LEDs to indicate whether the device 201 is in an on state or an off state (e.g., one or more of the LEDs may be illuminated by the controller when the device is in an on state).

The device 201 comprises a further input means (i.e., in addition to the button 212) in the form of a puff sensor 225. The puff sensor 225 is configured to detect a user drawing (i.e., inhaling) at the downstream end 218 of the consumable 202. The puff sensor 225 may, for example, be in the form of a pressure sensor, a flowmeter, or a microphone. The puff sensor 225 is operatively connected to the controller 208 in the electronics cavity 224, such that a signal from the puff sensor 225, indicative of a puff state (i.e., drawing or not drawing), forms an input to the controller 208 (and can thus be responded to by the controller 208).

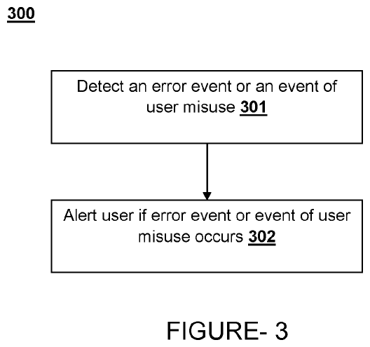

FIG. 3 illustrates flowchart of a method for detecting an occurrence of an error event or an event of user misuse.

As illustrated in FIG. 3, the method 300 includes one or more blocks implemented by the controller 208 of the device 201. The method 300 may be described in general context of controller executable instructions. Generally, controller executable instructions may include routines, programs, objects, components, data structures, procedures, modules, and functions, which perform particular functions or implement particular abstract data types.

The order in which the method 300 is described is not intended to be construed as a limitation, and any number of the described method blocks can be combined in any order to implement the method 300. Additionally, individual blocks may be deleted from the method 300 without departing from the scope of the subject matter described herein. Furthermore, the method 300 can be implemented in any suitable hardware, software, firmware, or combination thereof.

At block 301, the controller 208 is configured for detecting an error event and an event of user misuse of the device 201. In an aspect, detecting discussed at block 301 may comprise detecting both (i) error event and (ii) the event of user misuse, one at a time or simultaneously.

At block 302, the output device 110 is configured to alert the user that one of the error event or the user misuse event has occurred. In particular, upon detecting the occurrence of the error event or the user misuse event, as disclosed in block 301, the controller 208 is configured to provide a signal to the output device 111. In response to said signal, the output device 111 is configured to generate an alert indicating that an error event or user misuse event has occurred.

Although not explicitly disclosed in the flowchart, the output device 111 may be configured to generate one of visual, audio, and haptic feedback to alert the user of the error event or event of user misuse. To achieve this the output device 111 may include a plurality of LEDs, speaker or haptic feedback device embedded therein.

### Conclusion

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilized for realizing the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the words “have”, “comprise”, and “include”, and variations such as “having”, “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means, for example, +/−10%.

The words “preferred” and “preferably” are used herein refer to embodiments of the invention that may provide certain benefits under some circumstances. It is to be appreciated, however, that other embodiments may also be preferred under the same or different circumstances. The recitation of one or more preferred embodiments therefore does not mean or imply that other embodiments are not useful and is not intended to exclude other embodiments from the scope of the disclosure, or from the scope of the claims.

1-224. (canceled)

225. A smoking substitute device, comprising:
a controller; and
an output device;
wherein the controller is configured to detect an event of user misuse of the device, wherein the event of user misuse comprises one or more of: (a) physical tampering with a cap of the device (201); (b) tampering with a user interface of the device; and (c) inappropriate selection of a mode of the device including an attempt to switch the device into a high temperature mode when the ambient temperature is determined by the controller to be above a predetermined threshold temperature; characterized in that the controller (208) is also configured to detect an error event of the device whereby, upon detecting an occurrence of said error event or event of user misuse, the controller is configured to alert the user of said error event or event of user misuse via the output device, wherein the output device is configured to provide distinct alerts for each of an error event and a user misuse event, respectively.

226. The device according to claim 225, wherein the event of user misuse comprises at least one of:
lifting a cap of the device when the device is in use; and
one or more attempts to unlock the device with an incorrect user input, as determined by the controller by comparing the user input with a predetermined unlock pattern.

227. The device according to claim 225, wherein the error event comprises at least one of:
an electrical short-circuit;
an electrical open-circuit;
a microcontroller error;
an overheating of one or more components of the device;
a detection of ambient temperature determined by the controller to be above a predetermined threshold temperature;
a sensor error; and
a user interface error.

228. The device according to claim 227, wherein the error event comprises at least one of: short circuit on a heater, open circuit detection, microcontroller error, battery overheat, overheating of a heater, ambient temperature too high or too low, puff sensor error and user interface error.

229. The device according to claim 225, wherein the controller is coupled to a heater and is configured to disable the heater in response to detecting an occurrence of one or more error events or one or more events of user misuse.

230. The device according to claim 225, wherein the output device is configured to generate one or more of visual, audio, and haptic feedback to alert the user to the error event or the event of user misuse.

231. The device according to claim 225, wherein the controller is coupled to a memory configured to store data generated by the error event or event of user misuse.

232. The device according to claim 231, wherein the device is connectable to a further device to which the data generated by the error event or event of user misuse is transferable.

233. A method of operating a smoking substitute device, the method comprising:
detecting an occurrence of an error event or an event of user misuse; and
alerting the user to said error event or event of user misuse, wherein the event of user misuse comprises one or more of:
physical tampering with a cap of the device;
tampering with a user interface of the device; and
inappropriate selection of a mode of the device including an attempt to switch the device into a high temperature mode when the ambient temperature is determined by the controller to be above a predetermined threshold temperature; characterized in that:
said smoking substitute device is configured to detect an error event of the device and an event of user misuse of the device; and
wherein the method further comprises providing distinct alerts for each of an error event and a user misuse event, respectively.

234. The method according to claim 233, wherein the event of user misuse comprises at least one of:
lifting a cap of the device when the device is in use; and
one or more attempts to unlock the device with an incorrect user input, as determined by the controller by comparing the user input with a predetermined unlock pattern.

235. The method according to claim 233, wherein the error event comprises at least one of: short circuit on a heater, open circuit detection, microcontroller error, battery overheat, overheating of a heater, ambient temperature too high or too low, puff sensor error and user interface error.

236. The method according to claim 233, wherein the method further comprises disabling the heater of the device when the occurrence of an error event or an event of user misuse is detected.

237. The method according to claim 233, wherein alerting comprises generating one of visual, audio, or haptic feedback to alert the user of the error event or event of user misuse.

238-265. (canceled)
